(19) United States
(12) Patent Application Publication
(10) Pub. No.: US 2018/0055353 A1
(43) Pub. Date: Mar. 1, 2018

(54) METHOD AND APPARATUS FOR DETECTING THE VIEWING DIRECTION OF A PERSON

(71) Applicant: Robert Bosch GmbH, Stuttgart (DE)

(72) Inventor: Andreas Voelkel, Ispringen (DE)

(21) Appl. No.: 15/556,969

(22) PCT Filed: Jan. 11, 2016
(86) PCT No.: PCT/EP2016/050356

§ 371 Date: Sep. 8, 2017

(30) Foreign Application Priority Data
Mar. 10, 2015 (DE) 10 2015 204 283.9

(51) Int. Cl. A61B 3/113; G03B 13/02

(52) U.S. Cl. CPC A61B 3/113; G03B 2213/025; G03B 13/02

(57) ABSTRACT

A method for detecting the viewing direction of a person having a visual aid includes providing at least one optical property of a visual aid of the person, observing at least one eye of the person, and correcting data ascertained from the observation based on the at least one optical property of the visual aid. An apparatus designed to detect the viewing direction of a person includes an observation device set up to observe at least one eye of the person, a data record containing at least one optical property of a visual aid of the person, and a computing device equipped to correct the observation based on the data record.

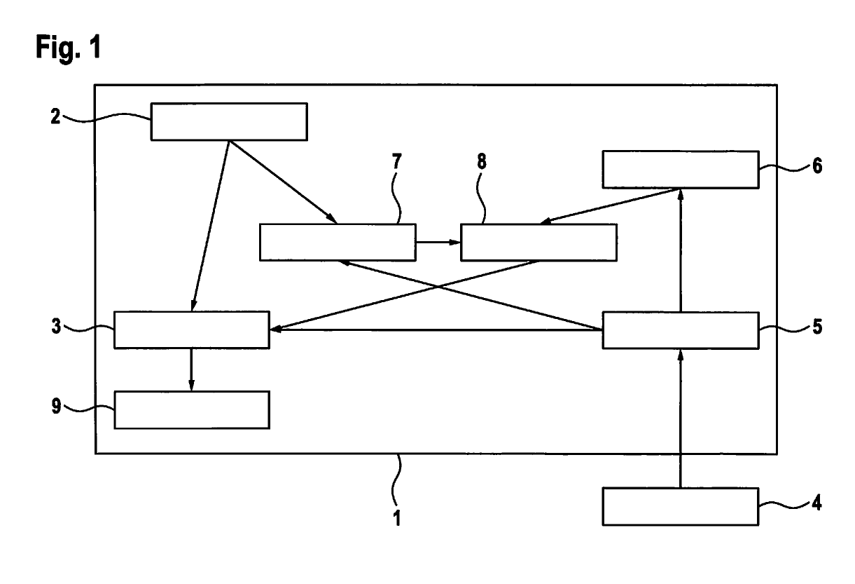

### CROSS-REFERENCE TO RELATED APPLICATIONS

The present application is the national stage of International Pat. App. No. PCT/EP2016/050356 filed Jan. 11, 2016, and claims priority under 35 U.S.C. §119 to DE 10 2015 204 283.9, filed in the Federal Republic of Germany on Mar. 10, 2015, the content of each of which are incorporated herein by reference in their entireties.

### FIELD OF THE INVENTION

The present invention relates to a method for detecting the viewing direction of a person, an apparatus equipped to detect the viewing direction of a person, as well as a computer-program product.

### BACKGROUND

Viewing-direction detection systems, for example, of vehicles or electronic devices, e.g., entertainment electronics or consumer electronics, usually detect the position and size of the pupil of the person observed.

### SUMMARY

Systems of this kind do not allow for possibly existing visual aids such as glasses or contact lenses, for example. Depending on the strength and geometry of the visual aid, an incorrect interpretation of the detected pupil position and pupil size may therefore come about in the viewing angle, since consequential viewing distortions owing to the visual aid used are not considered.

According to an example embodiment of the present invention, a method is provided for detecting the viewing direction of a person with a visual aid, the method including the following steps: providing at least one optical property of a visual aid of the person; observing at least one eye of the person; and correcting data ascertained from the observation based on the at least one optical property of the visual aid.

An advantage of the method is an increase of the robustness of the viewing-direction detection, as many interpretations of the image information occasioned by optical distortions of a visual aid can be avoided. Consequently, the gaze control becomes available to a larger range of users than is possible presently because of the fixed distortion tolerances of the image information.

To correct the data from the observation, at least one image can be taken, and the image can be processed in light of the at least one optical property. This imaging method can be based on one or more individual images or on a sequence of images such as a video film or video stream, for example. This image processing or image editing has the advantage that numerous already-proven algorithms are available.

To correct the data from the observation, an analysis can be carried out using the at least one optical property, allowing for limiting the analysis or processing to certain parts or areas, such as the area of the visual aid, for example. In this manner, it is possible to save on computing power.

The at least one optical property can include at least one of the following parameters: refraction, strength, sphere, axis, and cylinder. This at least one optical property is well-suited to describe the optical properties of the visual aid and to permit a suitable correction based thereon.

The at least one optical property can be converted into a model, a parameter field, a vector, and/or a matrix. These approaches allow a simple correction of the observed or detected eye of the person by an interaction of image or data record with the model, parameter field, vector, and/or matrix. For example, the optical properties can be converted into an imaging vector or an imaging matrix, which is then applied to the acquired data of the eye, to thus eliminate the distortion produced by the visual aid.

The correction can be employed at least during a calibration phase of the detection. If an initial calibration is provided in the case of the viewing-direction detection, the correction can be carried out directly in this phase, thereby reducing the later computing expenditure. In spite of the correction in a calibration phase, it can also be provided to additionally make further correction during the actual detection in order to increase the accuracy, for example.

According to an example embodiment of the present invention, an apparatus for detecting the viewing direction of a person includes an observation device set up to observe at least one eye of the person, a data record containing at least one optical property of a visual aid of the person, and a computing device equipped to correct the observation based on the data record.

An apparatus can include an electrical device or control unit that processes sensor signals and outputs control signals as a function thereof. An apparatus can also include one or more assistance systems of a vehicle. The same advantages and modifications hold true as described above.

The apparatus can be part of a driver-observation system of a vehicle. The vehicle can be a motor vehicle, especially a road-going vehicle, e.g., an automobile, a truck or a two-wheeled vehicle. The apparatus offers itself especially for vehicles, since very precise detection of the viewing direction of a driver of a vehicle may contribute decisively to traffic safety.

The apparatus can be part of an electronic device. The improved detection of the viewing direction can also promote increased spread or acceptance in the case of electronic devices like, for example, from the field of entertainment electronics.

A control unit according to the present invention for a motor vehicle is equipped to carry out a method described above. The integration into a control unit of a motor vehicle allows rapid and reliable access to the necessary data, and rapid correction targeted for the concerns in road traffic.

The present invention also proposes a computer-program product having computer-executable program code for carrying out the method above when the program is executed on an apparatus. The same advantages and modifications hold true as described above.

Exemplary embodiments of the present invention are explained more fully on the basis of the drawing and the following description.

### BRIEF DESCRIPTION OF THE DRAWINGS

The FIGURE is a schematic representation of a method and an apparatus for detecting the viewing direction of a person having a visual aid.

### DETAILED DESCRIPTION

The FIGURE shows a system or an apparatus 1 for detecting the viewing direction of a person. Apparatus 1 includes an observation device 2, for example, in the form of one or more driver-observation cameras. Observation device 2 takes individual images or sequences of images and makes them available to a computing device 3 such as a control unit of an assistance system, for instance. The image(s) of observation device 2 is/are evaluated in computing device 3. To that end, first of all, one or both pupils can be pinpointed in the data of observation device 2, and subsequently, a viewing-direction vector, for example, can be formed from the position and size of the pupil or pupils. This viewing-direction vector indicates the direction in which the observed person is looking. It is also possible to indicate on what or at what distance the gaze of the person is focused.

If the observed person is using a visual aid such as glasses or contact lenses, for example, from the perspective of observation device 2, the eye, or rather, the pupil is situated behind the visual aid. Therefore, the optical path of observation device 2 runs through the lens system of the visual aid, so that the eye, that is, the pupil is distorted subject to the optical properties of the visual aid.

In order to permit correct detection of the viewing direction in spite of the visual aid used, first of all, the properties, that is, the optical properties of the visual aid of the person are determined in an external system 4. This external system, which, for example, is used at an eye doctor or optician or the fabricator of the visual aid, generates a data record 5 having at least one optical property of the visual aid. This data record 5 is made available to apparatus 1. Data record 5 can be transmitted in wireless or wired fashion, e.g., with the aid of a smart phone, USB stick, e-mail or SMS from external system 4 to apparatus 1.

In an example, a driver observation system creates a driver data record 6 that is used primarily to identify the driver, and moreover, can be used for expanded functions such as comfort functions, for instance. Optionally, data record 5 having the optical properties of the visual aid is transferred to driver data record 6 and integrated into it. In this way, the optical properties of the visual aid are assigned exactly to the person who is wearing this visual aid. In an example, data record 5, or rather, the optical property includes at least one of the following parameters:

refraction, strength, sphere, axis, and cylinder. The visual aid can be described optically based on these values or parameters.

The driver observation system can include a calibration function 7, which, in an example, can be invoked upon the first contact of the person with apparatus 1. Calibration function 7 is able to display optical features whose position is known to apparatus 1, and to prompt the person to focus his gaze on these features. By observing, detecting, and/or recording the person, or rather the eyes and/or the pupils of the person when looking at the known marking, it is thus possible to calibrate apparatus 1 to the specific person. To that end, the optical properties, that is, data record 5 can be provided to calibration function 7, so that the optical properties of the visual aid of the person are already taken into account during the calibration.

Calibration function 7 generates a viewing-direction calibration data record 8, in which the specific calibration data are contained. Data from driver data record 6 can also be incorporated in this viewing-direction calibration data record 8. Correspondingly, the optical properties of the visual aid, that is, data record 5 can be integrated via driver data record 6 and/or via calibration function 7 into viewing-direction calibration data record 8. The optical properties of the visual aid are made available to computing device 3 either directly via data record 5 or indirectly via viewing-direction calibration data record 8. The data coming from observation device 2 are corrected in computing device 3 based on at least one optical property of the visual aid, so that the optical distortions resulting from the visual aid are eliminated algorithmically. This can already be carried out in a calibration phase and/or in the observation operation.

To that end, computing device 3 is able to calculate a model, a parameter field, a vector and/or a matrix from data record 5, that is, from the at least one optical property of the visual aid. The data from observation device 2 are used as input quantities for the construct selected in each case, or the selected construct is applied to the data of observation device 2. In the case of a model, the data from observation device 2 are used as input quantities, and the output quantity of the model is a representation from observation device 2 from which distortion has been eliminated. Via a parameter field, the processing of the data from observation device 2 can be parameterized, e.g., as filter settings. A vector or a matrix can be applied as optical imaging or transformation to the data of observation device 2.

The approaches described can be used on the complete data, that is, the entire image from observation device 2. In order to reduce computing expenditure or to increase the processing speed, it is possible to process only certain areas. To that end, certain portions like, for example, the pupils, the eyes, a certain region around the pupils, and/or eyes or the spatial extension of the visual aid can be used to thus define the areas of the observation, that is, of the data or of the image from observation device 2.

The model, the parameter field, the vector, and/or the matrix is/are then applied to this area. The result is a robust viewing direction, corrected in real time, which can be made available to a further system 9, more specifically, a software module, or a software routine. The viewing direction can also be further processed in computing device 3.

Although the present invention was described in detail and illustrated more fully through preferred exemplary embodiments, the invention is not limited by the disclosed examples, and one skilled in the art is able to derive other variations from it without departing from the scope of protection of the invention.

1-11. (canceled)

12. A method for detecting a viewing direction of a person having a visual aid, the method comprising:
obtaining, by a processor, at least one optical property of a visual aid of the person;
obtaining, by the processor, data ascertained from an observation of at least one eye of the person; and
correcting, by the processor, the ascertained data based on the at least one optical property of the visual aid.

13. The method of claim 12, further comprising obtaining at least one image, wherein the correcting includes processing the at least one image based on the at least one optical property.

14. The method of claim 12, wherein the correcting includes analyzing at least a portion of the data based on the at least one optical property and correcting the data based on a result of the analysis.

15. The method of claim 12, wherein the at least one optical property includes at least one of the following parameters: refraction, strength, sphere, axis, and cylinder.

16. The method of claim 12, further comprising converting the at least one optical property into at least one of a model, a parameter field, a vector, and a matrix, wherein the correcting is performed using the at least one of the model, parameter field, vector, and matrix.

17. The method of claim 12, wherein the correction is employed at least during a calibration phase of the detection.

18. An apparatus configured to detect the viewing direction of a person, the apparatus comprising:
a sensor configured to observe at least one eye of the person; and
a processor, wherein the processor is configured to:
obtain a data record containing at least one optical property of a visual aid of the person; and
correct data of the observation by the sensor based on the data record.

19. The apparatus of claim 18, wherein the apparatus is part of a driver-observation system of a vehicle.

20. The apparatus of claim 18, wherein the apparatus is part of an electronic device.

21. A control unit for detecting a viewing direction of a person having a visual aid, the control unit comprising:
a processor interfacing with a sensor, wherein the processor is configured to perform a method, the method including:
obtaining at least one optical property of a visual aid of the person;
obtaining from the sensor data ascertained from an observation of at least one eye of the person; and
correcting the ascertained data based on the at least one optical property of the visual aid.

22. A non-transitory computer-readable medium on which are stored instructions that are executable by a processor and that, when executed by the processor, cause the processor to perform a method for detecting a viewing direction of a person having a visual aid, the method comprising:
obtaining at least one optical property of a visual aid of the person;
obtaining data ascertained from an observation of at least one eye of the person; and
correcting the ascertained data based on the at least one optical property of the visual aid.
